(19) United States
(12) Patent Application Publication
(10) Pub. No.: US 2026/0051374 A1
(43) Pub. Date: Feb. 19, 2026

(54) COMPOSITE DESIGN APPARATUS AND METHOD BASED ON MULTISCALE SIMULATION

(71) Applicant: Foundation of Soongsil University-Industry Cooperation, Seoul (KR)

(72) Inventors: Ji Woong Kim, Seoul (KR); Myung Jae Kim, Seoul (KR); Jin Yong Lee, Seoul (KR); Hyo Kyeong Kim, Seoul (KR); Jong Wook Kwak, Seoul (KR); Ji Ho Kim, Seoul (KR)

(73) Assignee: Foundation of Soongsil University-Industry Cooperation, Seoul (KR)

(21) Appl. No.: 19/285,361

(22) Filed: Jul. 30, 2025

(30) Foreign Application Priority Data
Aug. 14, 2024 (KR) 10-2024-0109060

(51) Int. Cl. G16C 60/00; G06F 30/23; G06F 119/08

(52) U.S. Cl. CPC G16C 60/00; G06F 30/23; G06F 2119/08

(57) ABSTRACT

A composite design apparatus based on multiscale simulation includes a property calculation unit configured to calculate properties of a composite based on a density functional theory (DFT) method; a model generation unit configured to construct a simulation model for predicting thermal behavior of the composite under temperature conditions using the calculated properties as input; an evaluation unit configured to evaluate mechanical and thermal properties under temperature conditions through a simulation using the simulation model; and a result derivation unit configured to derive an optimal composite from the simulation results and predict a change in properties of the composite.

### CROSS-REFERENCE TO RELATED APPLICATION

This application claims the benefit under 35 U.S.C. § 119 (a) of Korean Patent Application No. 10-2024-0109060, filed on Aug. 14, 2024, the entire disclosure of which is incorporated herein by reference for all purposes.

### BACKGROUND

### 1. Field

The present disclosure relates to a composite design apparatus and method based on multiscale simulation.

### 2. Description of Related Art

Material discovery is a field of research focused on identifying new materials suitable for specific purposes. With a vast number of elemental substances available and an even greater variety of composites that can be formed by combining them, the search for a crystal structure that satisfies multiple target properties simultaneously demands extensive expertise, as well as significant time and cost.

Related prior art includes Korean Patent No. 10-0783331.

### SUMMARY

The present disclosure is directed to providing a composite design apparatus and method based on multiscale simulation, which are capable of minimizing the time and cost consumed in the compositional screening process by predicting the properties of a composite through multiscale simulation and deriving optimal conditions for the composite.

In one general aspect, composite design apparatus based on multiscale simulation includes: a property calculation unit configured to calculate properties of a composite based on a density functional theory (DFT) method; a model generation unit configured to construct a simulation model for predicting thermal behavior of the composite under temperature conditions using the calculated properties as input; an evaluation unit configured to evaluate mechanical and thermal properties of the composite under temperature conditions based on a simulation using the simulation model; and a result derivation unit configured to derive a composite material forming an optimal composite from the simulation results, and to predict changes in the properties of the composite.

The property calculation unit may be configured to compute the properties based on Coulomb interactions between electrons and nuclei constituting the composite, using the density functional theory method.

The property calculation unit may be configured to calculate the properties including density, coefficient of thermal expansion, Young's modulus, and Poisson's ratio.

The evaluation unit may be configured to derive mechanical and thermal properties of the composite based on a finite element analysis technique.

The thermal properties may include a coefficient of thermal expansion and thermal stress.

The result derivation unit may be configured to identify, as an optimal composite, a composite that exhibits a greater difference in coefficient of thermal expansion and thermal stress between a matrix and particles forming the composite, compared to other composites.

In another general aspect, a composite design method based on multiscale simulation includes: calculating, by a property calculation unit, properties of a composite based on density functional theory (DFT); constructing, by a model generation unit, a simulation model for predicting thermal behavior of the composite under temperature conditions, using the calculated properties as input; evaluating, by an evaluation unit, mechanical and thermal properties under temperature conditions through a simulation using the simulation model; and deriving, by a result derivation unit, a composite material suitable for forming an optimal composite from the simulation results, and predicting a change in properties of the composite.

The calculating of the properties based on the density functional theory may include computing the properties from Coulomb interactions between electrons and nuclei constituting the composite.

The calculating of the properties may include computing the properties including density, coefficient of thermal expansion, Young's modulus, and Poisson's ratio.

The evaluating of the mechanical and thermal properties may include deriving mechanical and thermal properties of the composite based on a finite element analysis technique.

The thermal properties may include a coefficient of thermal expansion and thermal stress.

The result derivation unit may be configured to identify, as an optimal composite, a composite that exhibits a greater difference in coefficient of thermal expansion and thermal stress between a matrix and particles forming the composite, compared to other composites.

According to an embodiment of the present invention, there is provided a composite design apparatus and method based on multiscale simulation, which allows minimizing the time and cost consumed in the compositional screening process by predicting the properties of a composite through multiscale simulation and deriving optimal conditions for the composite.

### BRIEF DESCRIPTION OF THE DRAWINGS

FIG. 1 is a diagram illustrating the configuration of a composite design apparatus based on multiscale simulation according to an embodiment of the present disclosure.

FIG. 2 is a diagram illustrating an example of property calculation in the composite design apparatus based on multiscale simulation according to an embodiment of the present disclosure.

FIG. 3 is a diagram illustrating an example of DFT computation in the composite design apparatus based on multiscale simulation according to an embodiment of the present disclosure.

FIG. 4 is a diagram illustrating an example of finite element analysis in the composite design apparatus based on multiscale simulation according to an embodiment of the present disclosure.

FIG. 5 is a diagram illustrating an example of element discretization for finite element analysis in the composite design apparatus based on multiscale simulation according to an embodiment of the present disclosure.

FIG. 6 is a diagram illustrating an example of thermal property results in the composite design apparatus based on multiscale simulation according to an embodiment of the present disclosure.

FIG. 7 is a diagram illustrating the flow of a composite design method based on multiscale simulation according to an embodiment of the present disclosure.

### DETAILED DESCRIPTION

The objectives and advantages of the present disclosure, as well as the technical configurations for achieving them, will become more apparent from the following detailed description of embodiments with reference to the accompanying drawings. In the description that follows, detailed explanations of known functions or configurations may be omitted when they are deemed to unnecessarily obscure the essence of the disclosure.

It should be understood, however, that the present disclosure is not limited to the specific embodiments described herein. Rather, it includes all modifications, equivalents, and substitutions that fall within the spirit and scope of the disclosure.

The terms used herein are defined in consideration of the functions of the disclosure and may vary depending on the context, the intent of the user, or customary usage.

The disclosure is not limited to the embodiments disclosed below and may be implemented in various other forms. The embodiments are provided merely to fully convey the scope of the disclosure to those skilled in the art. The disclosure is defined only by the claims, and the meaning of the claims should be interpreted in light of the entire specification.

The terminology used in this application is intended solely to describe particular embodiments and is not meant to limit the disclosure. Unless clearly indicated otherwise by the context, the singular form also includes the plural. Additionally, terms such as “include” and “have” are intended to indicate the presence of stated features, steps, components, or combinations thereof, but do not preclude the presence or addition of one or more other features, steps, components, or combinations thereof.

Unless otherwise defined, all terms used herein, including technical and scientific terms, have the same meaning as would be commonly understood by one of ordinary skill in the art to which this disclosure pertains. Terms defined in commonly used dictionaries should be interpreted consistently with their ordinary meanings in the relevant technical field and should not be interpreted in an overly idealized or formal sense unless expressly defined otherwise in this disclosure.

Preferred embodiments of the present disclosure will now be described in detail with reference to the accompanying drawings. In the drawings, the same reference numerals are used for identical or corresponding components, and redundant descriptions of such components may be omitted for clarity.

In the case of composite design, research is typically conducted using empirical methods, which exhibit very low efficiency in terms of both time and cost. In composites, factors such as the coefficient of thermal expansion, thermal stress, and yield strength have a significant impact on property evaluation, and these relationships can be expressed as shown in Equation 1 below.

$$σ = E ϵ = E α Δ T \qquad ( Equation 1 )$$

$$σ : thermal stress , E : elastic modulus , ϵ : strain ,$$

$$α = coefficient of thermal expansion , Δ T : temperature change$$

Accordingly, as the difference between the elastic modulus and the coefficient of thermal expansion increases, the thermal stress also increases.

In prior research, there have been attempts to investigate the strength characteristics of aluminum alloy A356 by incorporating Al₂O₃ and ScF₃ nanoparticles to examine the coefficient of thermal expansion, thermal stress, and yield strength. According to such studies, not only does testing high-temperature properties require substantial cost, but there also exist disadvantages in that the physical and mechanical properties of the nanoparticle compositions are difficult to clearly understand, and compositions other than Al₂O₃ and ScF₃ cannot be effectively explored.

Accordingly, the present disclosure relates to a composite design apparatus based on multiscale simulation, which overcomes the limitations of conventional structure material R&D—where empirical knowledge and traditional methods play a dominant role—by leveraging digital technologies through the application of first principles and the finite element method.

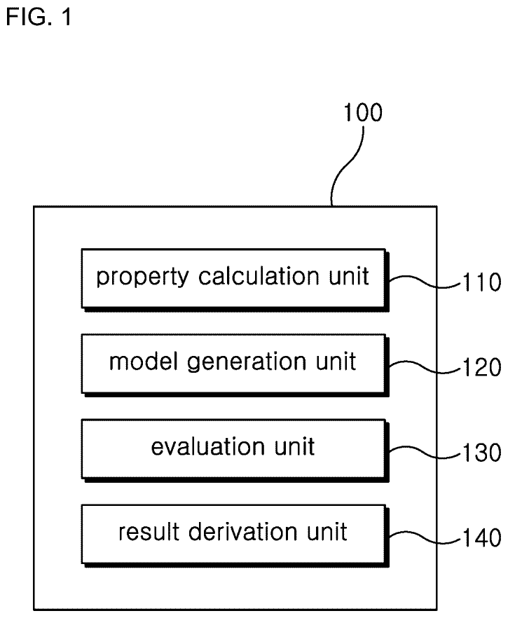

FIG. 1 is a diagram illustrating the configuration of a composite design apparatus based on multiscale simulation according to an embodiment of the present disclosure.

Referring to FIG. 1, the composite design apparatus based on multiscale simulation 100 may include a property calculation unit 110, a model generation unit 120, an evaluation unit 130, and a result derivation unit 140.

The property calculation unit 110 may calculate the properties of a composite based on a density functional theory (DFT) method, which is a first-principles calculation technique. The property calculation unit 110 may compute the material properties from the Coulomb interactions between electrons and nuclei constituting the composite, based on the density functional theory method.

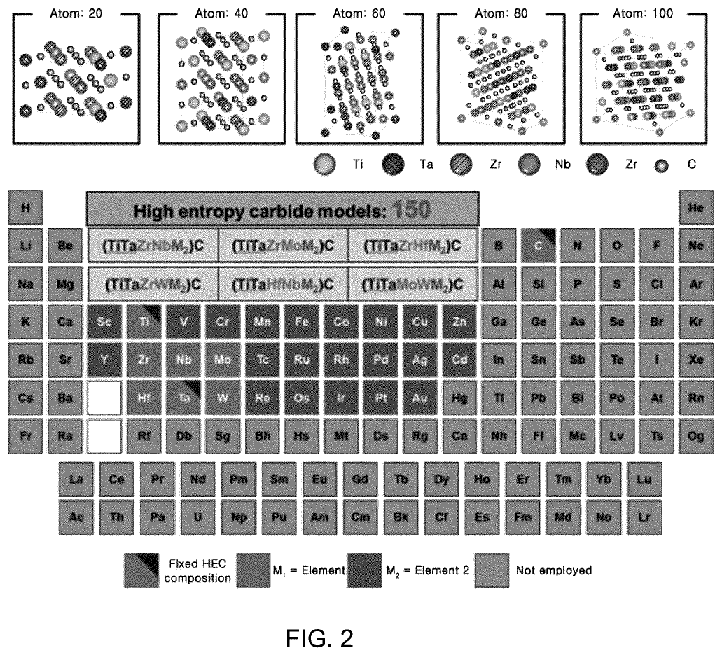

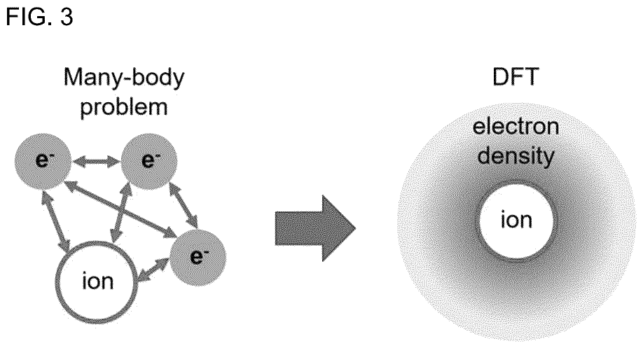

FIG. 2 is a diagram illustrating an example of property calculation in the composite design apparatus based on multiscale simulation according to an embodiment of the present disclosure. FIG. 3 is a diagram illustrating an example of DFT computation in the composite design apparatus based on multiscale simulation according to an embodiment of the present disclosure.

Referring to FIGS. 2 and 3, a property calculation unit 110 may calculate material properties at 0 K. Specifically, the property calculation unit 110 may predict material properties based on first-principles calculations without requiring experimental data, using only physical constants and quantum mechanical principles. In this case, the property calculation unit 110 may compute properties including, but not limited to, density, coefficient of thermal expansion, Young's modulus, and Poisson's ratio.

In other words, the property calculation unit 110 may overcome the inefficiency of conventional experimental methods by predicting the thermal properties of new composite compositions based on simulation. In particular, by calculating Coulomb interactions between electrons and nuclei based on DFT, the material properties of new composite compositions can be predicted without experimental data. Hereinafter, a simulation for predicting the thermal properties and physical characteristics of composites through finite element analysis will be described in detail.

A model generation unit 120 may generate a 3D model of a structure or material to be analyzed. Specifically, the model generation unit 120 may construct a simulation model that predicts the thermal behavior of the composite under temperature conditions by inputting mechanical and thermal properties derived from DFT calculations into the finite element analysis model. The mechanical properties may include, but are not limited to, elastic modulus, yield strength, and tensile strength.

The model generation unit 120 may construct the simulation model by generating a mesh that divides the model into small finite elements and by setting external loads, pressures, displacements, and boundary conditions to be applied to the model.

The simulation model may simulate the material properties of the composite by calculating the deformation, stress, and displacement of each element under various temperature conditions. That is, it may simulate and display how the properties of the composite change with temperature. The model generation unit 120 may construct a simulation model by configuring a thermal environment to simulate various temperature conditions, and may also construct simulation models that reflect variations in additive elements and compositions.

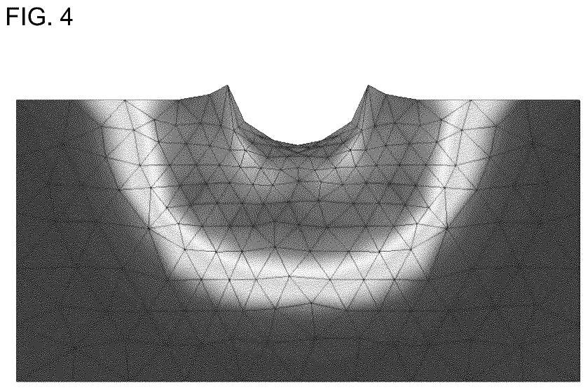

FIG. 4 is a diagram illustrating an example of finite element analysis in the composite design apparatus based on multiscale simulation according to an embodiment of the present disclosure.

An evaluation unit 130 may evaluate the mechanical and thermal properties of the composite under temperature conditions through simulation using the simulation model. That is, the evaluation unit 130 may analyze the simulation results to evaluate the mechanical behavior of a structure or material related to thermal stress, and may evaluate thermal behavior by calculating the temperature distribution and thermal expansion of each element. Referring to FIG. 4, the evaluation unit 130 may derive the mechanical and thermal properties of the composite based on a finite element analysis (FEA) technique. The finite element analysis technique is a numerical analysis method used to analyze the physical properties of materials, and is suitable for analyzing structural deformation, stress, heat transfer, and fluid dynamics.

Mechanical properties refer to the evaluation of how a material responds when subjected to mechanical forces such as external loads, pressure, or deformation. Key mechanical properties include elastic modulus, yield strength, tensile strength, ductility, hardness, and fracture toughness. These properties may be mainly used to predict thermal stress. Thermal properties refer to the evaluation of how a material responds to temperature changes, and key thermal properties may include the coefficient of thermal expansion and thermal conductivity.

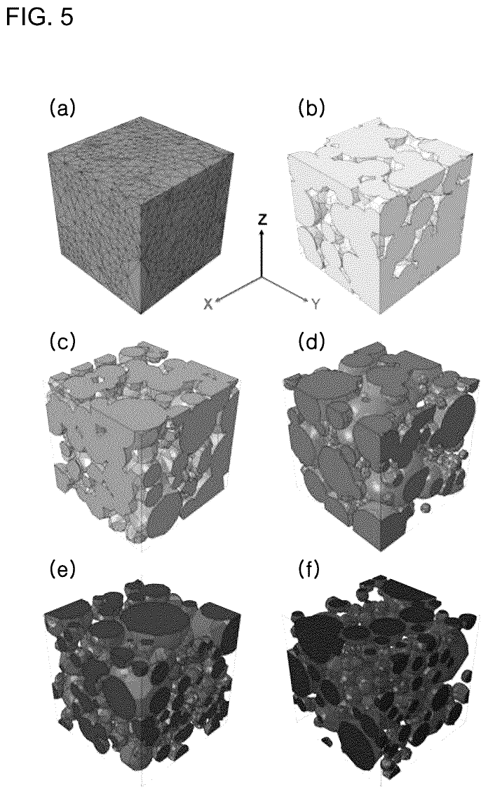

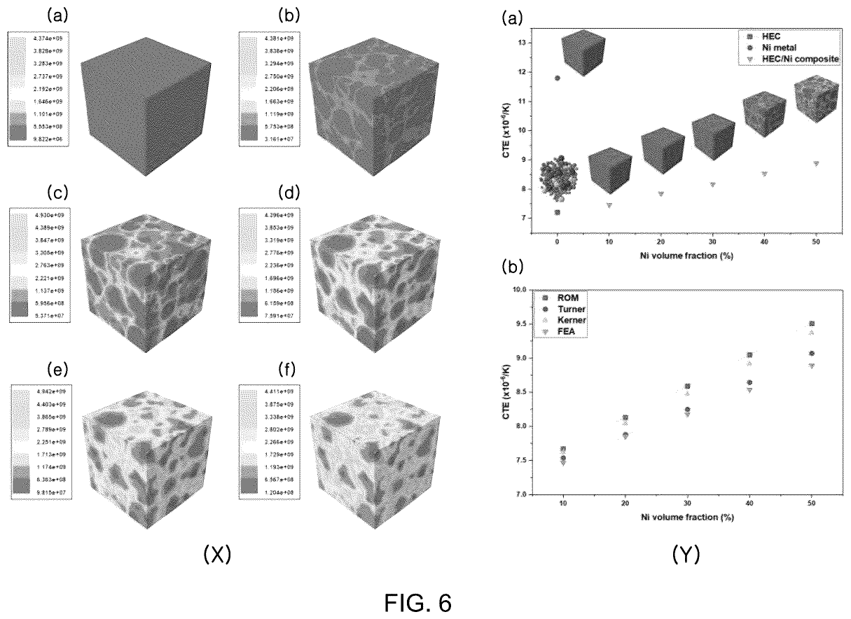

FIG. 5 is a diagram illustrating an example of element discretization for finite element analysis in the composite design apparatus based on multiscale simulation according to an embodiment of the present disclosure. FIG. 6 is a diagram illustrating an example of thermal property results in the composite design apparatus based on multiscale simulation according to an embodiment of the present disclosure.

Referring to FIG. 5, the evaluation unit 130 may divide the composite to be analyzed into a plurality of finite elements and calculate and evaluate the mechanical and thermal properties of each element through simulation.

Referring to FIG. 6, the evaluation unit 130 may model the thermal expansion of the composite according to temperature. By way of example, the thermal properties may include the coefficient of thermal expansion and thermal stress. (X) of FIG. 6 illustrates an example of FEM-based modeling of thermal expansion according to temperature, and (Y) of FIG. 6 illustrates a graph of the coefficient of thermal expansion.

A result derivation unit 140 may derive the optimal composite from the simulation results and predict changes in the properties of the composite. By simultaneously analyzing the microstructure and macroscopic properties of the composite through multiscale simulation, the result derivation unit 140 not only enables more precise prediction, but also allows the properties of the composite to be predicted in advance and the optimal conditions to be derived.

The result derivation unit 140 may identify a composite as optimal based on its comparatively high coefficient of thermal expansion and thermal stress relative to other evaluated composites.

In addition, the result derivation unit 140 may predict changes in the mechanical and thermal properties of the identified composite through finite element analysis, using mechanical properties such as Young's modulus, Poisson's ratio, and coefficient of thermal expansion—derived from first-principles calculations—as input values.

The composite design apparatus based on multiscale simulation 100 according to an embodiment of the present disclosure may study the characteristics of a material in various states using multiscale simulation. Specifically, it may examine fundamental structural properties, such as lattice constants and elastic constants for each composition of the material, through first-principles calculations. In addition, it may calculate various thermal and physical properties, including Poisson's ratio, Young's modulus, coefficient of thermal expansion, specific heat, thermal conductivity, and Debye temperature.

Based on the data obtained from first-principles calculations, the thermal and mechanical behavior of a material may be analyzed through finite element analysis. Finite element analysis may be performed using parameters such as density, Poisson's ratio, Young's modulus, and coefficient of thermal expansion, which are derived from the first-principles calculations.

Accordingly, the composite design apparatus based on multiscale simulation 100 may predict the properties of a composite prior to the manufacturing process and derive optimal conditions. As a result, it is possible not only to reduce the cost and time required for experimentation and production, but also to predict the optimal conditions of the composite, thereby securing both stability and functionality and minimizing the defect rate.

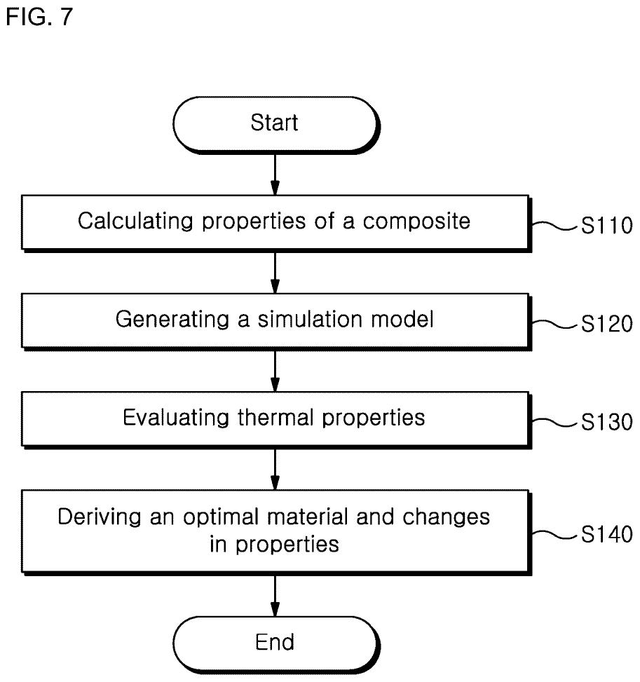

FIG. 7 is a diagram illustrating the flow of a composite design method based on multiscale simulation according to an embodiment of the present disclosure.

The composite design method based on multiscale simulation illustrated in FIG. 7 may be performed by the composite design apparatus based on multiscale simulation 100, as described with reference to FIGS. 1 to 6. Accordingly, even if certain details are omitted below, the features and operations described with respect to the apparatus 100 in FIGS. 1 to 6 are equally applicable to FIG. 7.

Referring to FIG. 7, in step S110, a property calculation unit 110 may calculate the properties of a composite based on a density functional theory (DFT) method, which is a type of first-principles calculation. The property calculation unit 110 may compute the material properties based on the Coulomb interactions between electrons and nuclei constituting the composite using the DFT method.

The property calculation unit 110 may calculate the properties of the composite at 0 K. Specifically, by calculating the material properties based on first-principles methods, the property calculation unit 110 may predict the properties without requiring experimental data, using only physical constants and quantum mechanical principles. In this process, the property calculation unit 110 may compute properties including, but not limited to, density, coefficient of thermal expansion, Young's modulus, and Poisson's ratio.

In step S120, a model generation unit 120 may construct a simulation model for predicting the thermal behavior of the composite under temperature conditions, using the calculated material properties as input. The simulation model may simulate the material properties of the composite under various temperature conditions. That is, it may simulate and display how the material properties of the composite change in response to temperature variations. The model generation unit 120 may construct the simulation model by creating a thermal environment for simulating different temperature conditions, and may also construct simulation models that reflect variations in additive elements and compositions.

In step 130, an evaluation unit 130 may evaluate the mechanical and thermal properties of the composite under temperature conditions through simulation using the simulation model. The evaluation unit 130 may derive the mechanical and thermal properties of the composite based on a finite element analysis (FEA) technique. The finite element analysis technique is a numerical analysis method used to analyze the physical properties of materials and is suitable for analyzing structural deformation, stress, heat transfer, and fluid dynamics.

The evaluation unit 130 may divide the composite to be analyzed into a plurality of finite elements and calculate and evaluate the mechanical and thermal properties of each element through simulation. The evaluation unit 130 may model the thermal expansion of the composite according to temperature. By way of example, the thermal properties may include the coefficient of thermal expansion and thermal stress.

In step S140, a result derivation unit 140 may derive the optimal composite from the simulation results and predict changes in the properties of the composite. The result derivation unit 140 not only enables more precise prediction by simultaneously analyzing the microstructure and macroscopic properties of the composite through multiscale simulation, but also allows the properties of the composite to be predicted in advance and optimal conditions to be derived. The result derivation unit 140 may identify a composite as optimal when the differences in coefficient of thermal expansion and thermal stress among composites are large. In addition, the result derivation unit 140 may predict changes in the mechanical and thermal properties of the identified composite through finite element analysis, using mechanical properties-such as Young's modulus, Poisson's ratio, and coefficient of thermal expansion-derived from first-principles calculations as input values.

The composite design method based on multiscale simulation according to an embodiment of the present disclosure may be implemented in the form of program instructions executable through various computer means and recorded on a computer-readable medium. The computer-readable medium may include program instructions, data files, data structures, or combinations thereof. The program instructions recorded on the medium may be specifically designed and configured for the present disclosure, or they may be known and available to those skilled in the field of computer software. Examples of computer-readable recording media include magnetic media such as hard disks, floppy disks, and magnetic tapes; optical media such as CD-ROMs and DVDs; magneto-optical media such as floptical disks; and hardware devices specially configured to store and execute program instructions, such as ROM, RAM, and flash memory. The program instructions may include not only machine code generated by compilers but also high-level language code that can be executed by computers using interpreters or the like. The above-described hardware devices may be configured to operate as one or more software modules for executing the operations of the present disclosure, and vice versa.

The features, structures, and effects described in the above embodiments are included in at least one embodiment of the present disclosure and are not necessarily limited to only one embodiment. Furthermore, the features, structures, and effects exemplified in each embodiment may be combined or modified with respect to other embodiments by those skilled in the art to which the embodiments pertain.

Accordingly, such combinations and modifications should be construed as falling within the scope of the present disclosure. While the foregoing description has been made primarily with reference to specific embodiments, it is merely illustrative and not intended to limit the scope of the present disclosure. It will be apparent to those skilled in the art that various modifications and applications not specifically exemplified above are possible without departing from the essential characteristics of the present disclosure. For example, each component specifically illustrated in the embodiments may be implemented in a modified form. Any differences relating to such modifications and applications should also be construed as being included within the scope of the present disclosure as defined by the appended claims.

What is claimed is:

1. A composite design apparatus based on multiscale simulation, comprising:
a property calculation unit configured to calculate properties of a composite based on a density functional theory (DFT) method;
a model generation unit configured to construct a simulation model for predicting thermal behavior of the composite under temperature conditions using the calculated properties as input;
an evaluation unit configured to evaluate mechanical and thermal properties of the composite under temperature conditions based on a simulation using the simulation model; and
a result derivation unit configured to derive a composite material forming an optimal composite from the simulation results, and to predict changes in the properties of the composite.

2. The composite design apparatus of claim 1,
wherein the property calculation unit is configured to compute the properties based on Coulomb interactions between electrons and nuclei constituting the composite, using the density functional theory method.

3. The composite design apparatus of claim 2,
wherein the property calculation unit is configured to calculate the properties including density, coefficient of thermal expansion, Young's modulus, and Poisson's ratio.

4. The composite design apparatus of claim 1,
wherein the evaluation unit is configured to derive mechanical and thermal properties of the composite based on a finite element analysis technique.

5. The composite design apparatus of claim 1,
wherein the thermal properties include a coefficient of thermal expansion and thermal stress.

6. The composite design apparatus of claim 1,
wherein the result derivation unit is configured to identify, as an optimal composite, a composite that exhibits a greater difference in coefficient of thermal expansion and thermal stress between a matrix and particles forming the composite, compared to other composites.

7. A composite design method based on multiscale simulation, comprising:
calculating, by a property calculation unit, properties of a composite based on density functional theory (DFT);
constructing, by a model generation unit, a simulation model for predicting thermal behavior of the composite under temperature conditions, using the calculated properties as input;
evaluating, by an evaluation unit, mechanical and thermal properties under temperature conditions through a simulation using the simulation model; and
deriving, by a result derivation unit, a composite material suitable for forming an optimal composite from the simulation results, and predicting a change in properties of the composite.

8. The composite design method of claim 7,
wherein the calculating of the properties based on the density functional theory comprises computing the properties from Coulomb interactions between electrons and nuclei constituting the composite.

9. The composite design method of claim 7,
wherein the calculating of the properties comprises computing the properties including density, coefficient of thermal expansion, Young's modulus, and Poisson's ratio.

10. The composite design method of claim 7,
wherein the evaluating of the mechanical and thermal properties comprises deriving mechanical and thermal properties of the composite based on a finite element analysis technique.

11. The composite design method of claim 7,
wherein the thermal properties include a coefficient of thermal expansion and thermal stress.

12. The composite design method of claim 7,
wherein the result derivation unit is configured to identify, as an optimal composite, a composite that exhibits a greater difference in coefficient of thermal expansion and thermal stress between a matrix and particles forming the composite, compared to other composites.
